(19) United States
(12) Patent Application Publication
(10) Pub. No.: US 2009/0305974 A1
(43) Pub. Date: Dec. 10, 2009

(54) THERAPEUTIC INDICATIONS OF COLONY STIMULATING FACTORS

(75) Inventors: Alfred Bach, Edingen-Neckarhausen (DE); Hans-Jürgen Quadbeck-Seeger, Bad Durkheim (DE)

Correspondence Address: FOLEY AND LARDNER LLP;SUITE 500, 3000 K STREET NW, WASHINGTON, DC, 20007, US

(73) Assignee: SYGNIS Bioscience GmbH & Co., KG

(21) Appl. No.: 12/162,326

(22) PCT Filed: Jan. 24, 2007
(86) PCT No.: PCT/EP07/50685

§ 371 Date: Mar. 13, 2009

(30) Foreign Application Priority Data
Jan. 27, 2006 (DE) 10 2006 004 142.9

(51) Int. Cl. A61K 38/18; A61P 25/00

(52) U.S. Cl. 514/12

(57) ABSTRACT

The present invention relates to the use of at least one colony stimulating factor (CSF) for the production of medicinal products in the treatment or prophylaxis of coma or neurotoxicity.

The colony stimulating factors (CSF) are a group of regulatory proteins that are responsible for controlling the proliferation and differentiation of hematopoietic cells such as granulocytes, megakaryocytes and monocytes or macrophages. Without appropriate CSFs, these hematopoietic cells cannot survive and/or proliferate in culture. The CSFs belong to the cytokine group. Together with erythropoietin (EPO) and some interleukins, they form the group of hematopoietic growth factors.

The CSF group includes the factors M-CSF (macrophage-colony stimulating factor; also CSF-1), GM-CSF (macrophage/granulocyte-colony stimulating factor; also CSF-2), G-CSF (granulocyte-colony stimulating factor; also CSF-3) and multi-CSF (multifunctional colony stimulating factor; also IL3) according to their specificity with respect to the various hematopoietic cells. Purification and cloning of the individual CSFs makes molecular characterization possible. The aforesaid four CSFs are glycoproteins, but they do not display any homology at the level of the primary structure (amino acid sequence).

M-CSF is produced by monocytes, granulocytes, endothelial cells and fibroblasts. Furthermore, however, activated B- and T-cells, as well as a number of tumor cell lines are able to synthesize this factor. M-CSF is a homodimeric glycoprotein. The sugar moiety is not necessary for biological activity. There are several variants with different molecular weights, which result from alternative splicing of the RNA.

M-CSF promotes the proliferation and differentiation of hematopoietic stem cells to macrophages, but mainly the growth, differentiation and functional activity of monocytes. Whereas human M-CSF also exhibits activity in mouse and rat cells, the murine factor is inactive in human cells.

G-CSF is secreted by activated monocytes, macrophages and neutrophils, by stroma cells, fibroblasts and endothelial cells, and by various tumor cell lines (e.g. human bladder cancer cell line). Mature human G-CSF is a monomeric glycoprotein with 174 amino acids, where the sugar moiety is not necessary for biological activity. Another variant with 177 amino acids, resulting from alternative splicing of the RNA, displays greatly reduced biological activity.

G-CSF promotes the proliferation and differentiation of hematopoietic precursor cells to neutrophilic granulocytes and also activates these. Furthermore, G-CSF also acts as a mitogenic agent.

The most important clinical application of G-CSF is the treatment of leukopenia, e.g. following chemotherapy and/or radiotherapy.

GM-CSF is a monomeric glycoprotein of 127 amino acids, where the sugar moiety is not necessary for biological activity. The GM-CSF receptor occurs not only on hematopoietic cells, but also e.g. on endothelial cells.

The specificity of GM-CSF is generally less pronounced than, for example, that of G-CSF. Thus, GM-CSF stimulates the proliferation and differentiation of neutrophil, eosinophil and monocyte lines and activates their mature form. At low concentrations the factor exerts a chemotactic action on eosinophils and neutrophils. As GM-CSF is produced by the cells (T-lymphocytes, macrophages, endothelial cells and mast cells) that are involved in an inflammatory response, it can be assumed that this factor plays an important role as mediator in inflammation.

In synergy with EPO, GM-CSF also promotes the proliferation of erythroid and megakaryocyte precursor cells.

GM-CSF finds clinical application for reconstitution of hematopoiesis. Its most important use is for the treatment of neutropenia, e.g. in connection with chemotherapy or radiotherapy.

Multi-CSF is mainly produced by activated T-cells, but also by keratinocytes, NK cells, mast cells, endothelial cells and monocytes. Mature human multi-CSF is a glycoprotein of 133 amino acids, where the sugar moiety is not necessary for biological activity.

Multi-CSF has a very broad spectrum of biological activities. Thus, multi-CSF supports the proliferation and differentiation of nearly all types of hematopoietic precursor cells. As initial factor, it makes the hematopoietic stem cells responsive to later-acting factors such as EPO and GM-CSF. The biological activities of multi-CSF are species-specific.

In view of this promotion of proliferation, differentiation and activation of cells of the hematopoietic system, CSFs are used therapeutically for reconstitution of hematopoiesis. Accordingly, mainly recombinant G-CSF (e.g. filgrastim) is used therapeutically for the treatment of neutropenia following chemotherapy or radiotherapy.

Furthermore, yet more therapeutic applications are described for CSFs. In this connection we may mention e.g. the use of CSF for the treatment of infections (WO 88/00832) and for promoting wound healing (WO 92/14480). The observation that CSFs may also play an important role in angiogenesis (WO 97/14307) and particularly in arteriogenesis (WO 99/17798) offers the possibility of using these factors for the treatment of ischemias such as myocardial infarction and stroke, by restoring and/or improving the blood flow in the affected tissue.

It has also been observed that certain CSF receptors are also present on neurons (DE-A 100 33 219). Accordingly, a neuroprotective and neuroregenerative action was recently demonstrated for G-CSF for the treatment of focal cerebral ischemia in an animal model [Schabitz et al. Stroke. 34:745 (2003); Schneider et al. J Clin Invest. 115:2083 (2005)].

A condition for which there continues to be a large demand for suitable medicinal products is coma. Coma is a severe degree of disturbance of consciousness, in which the patient can no longer be woken by external stimuli (Pschyrembel, 259th Edition, 2002; pp. 603-604, pp. 882-883, p. 978, p. 1110, p. 1620). Depending on the symptoms and the causes, the following coma states are distinguished, among others:

Apallic syndrome (persistent vegetative state): a clinical picture that is included among the decerebration syndromes, with functional loss of the cerebral cortex, generally as a result of anoxia of the brain (e.g. after head injury, intoxication, shock or resuscitation) and disturbance of the ascending reticular activating system with retention of brainstem function. The patient is awake and his eyes are open, but he does not show any spontaneous and reactive movements and also no eye fixation. Also there are no spontaneous utterances. Spontaneous breathing and circulatory regulation are, however, intact. If caused by injury or infection, functional recovery is still possible after some months, though it is unlikely after more than three months. In the absence of remission, death occurs after two to five years (e.g. as a result of complications such as pneumonia, urinary tract infection or decubitus).

“Locked-in” syndrome: inability, although remaining conscious, to communicate spontaneously by speech or through movements. Communication through eye movements is possible. The cause is a bilateral transverse lesion of the tractus corticobulbaris and tractus corticospinalis in the region of the pons, e.g. in arteria basilaris thrombosis. The prognosis is unfavorable.

Akinetic mutism: mutism (dumbness) as a result of general inhibition of motor capabilities including facial expression, gestures and speech. Speech and movement do not take place spontaneously, and only slowly and with a delay after being requested. There is also disturbance of the sleeping-waking cycle. Pain stimuli increase vigilance and make limited contact possible. Consciousness is fully retained, and there may be amnesia. Akinetic mutism occurs e.g. after frontal lobe lesions, in psychoses, tumor or hemangioma near the 3rd ventricle of the brain or in the mesencephalon in arteria basilaris thrombosis or in encephalitis.

The Glasgow Coma Scale (GCS) permits quantitative classification according to the severity of the disturbance of consciousness (“mild disturbance of consciousness” (14-15 points); “moderate disturbance of consciousness” (13-9) points; “severe disturbance of consciousness” (3-8 points)). The patient's reaction is assessed in three areas (eye opening, motor behavior and speech) and the corresponding points achieved are added together.

Treatment of coma patients should mainly be directed towards increasing the probability of awakening and reducing or reversing the neurological damage caused by being in the comatose state. Initial studies show such a therapeutic effect e.g. for treatment with acetyl-L-carnitine (EP-A 0 498 144). At present, however, no commercial preparation that fulfils this therapeutic requirement is in clinical use.

Owing to their particular properties, neurons have increased sensitivity to the action of toxic substances (Anthony, Montine, Valentine & Graham; Toxicology; Ed. Casarett & Doull; 6th edition, 2001; pp. 535-563).

Thus, because of their high energy requirements, neurons are particularly dependent on aerobic metabolism. Even short interruptions in the supply of oxygen or glucose can damage the neurons. An example of this is hypoxia as a result of carbon monoxide poisoning, in which there is mainly damage to the neurons that are especially sensitive to this, such as those in certain regions of the cerebral cortex.

Another reason for the particular sensitivity of the neurons to toxic substances is the typical structure of the neurons with their long processes, the axons. These axons, the length of which can reach 200 000 times the diameter of the cell body, must be supplied from the cell body. The provision of protein synthesis machinery for such a large volume of cytoplasm and the axonal transport of the synthesis products make heavy demands on the neurons.

The special sensitivity of the cells of the nervous system is countered by a special protection, the blood-brain barrier. In contrast to the blood vessels of other organs, the cerebral capillary vessels are enveloped by their epithelial cells without any gaps (“tight junctions”). In addition, the surrounding glial cells form a further barrier to the passive transport of many substances as well as toxins. The substances that the nerve cells require from the blood are brought in through the barrier by active transport. Only lipid-soluble substances, and hence also toxins of that type, can penetrate the blood-brain barrier passively. The protection afforded by the blood-brain barrier has some other important limitations. Thus, certain regions of the nervous system, e.g. the circumventricular organ, are not protected by a blood-brain barrier. Moreover, the blood-brain barrier of neonates is not yet fully manifested.

Depending on the mechanism of neurotoxicity, a distinction is made between four different subgroups: neuronopathy, axonopathy, myelinopathy and transmission-associated toxicity.

A neuronopathy means there is primarily a lesion of the neuron cell body. The loss of a neuron is irreversible and includes the degradation of all cytoplasmic processes, such as dendrites and axons, and of the associated myelin. Various neurotoxins are specific for certain neuronal subpopulations and so can lead to characteristic losses of function. Doxorubicin for example, a cytostatic, which is deposited in the DNA double strand, mainly damages the neurons of the peripheral nervous system (PNS) and other nerve cells not protected by the blood-brain barrier. The neurotoxin MPTP (1-methyl-4-phenyl-1,2,3,6-tetrahydropyridine) can, in contrast, owing to its uncharged state, penetrate the blood-brain barrier. Then it is oxidized enzymatically to the corresponding pyridinium ion (MPP) and taken up via the dopamine-transport system mainly by the dopaminergic neurons of the substantia nigra. There, the MPP blocks the mitochondrial cellular respiration and so leads to the death of these neurons. The symptoms of this intoxication correspond to those of irreversible Parkinson's disease. Low doses of MPTP, which do not cause any acute symptoms, can increase the predisposition for Parkinson's disease. Other neurotoxins acting by the mechanism of neuronopathy include some heavy metals (e.g. lead, bismuth, mercury and manganese), some antibiotics (e.g. chloramphenicol and streptomycin) and alcohols (e.g. methanol and ethanol).

In the case of the neurotoxic diseases that come under the heading axonopathy, primarily the axon is damaged. Following the primary damage, generally there is degradation of the distal end of the axon in a multistage process, whereas the cell body of the neuron survives. Whereas this degradation in the central nervous system is irreversible, the axons of the peripheral nervous system can regenerate. n-Hexane and carbon disulfide are examples of neurotoxins that act in this way. They lead to crosslinking of the axonal neurofilaments, followed by swelling of the axons and impairment of neurofilament transport. Generally axonopathy leads to peripheral neuropathy. The sensory and motor impulse conduction is increasingly impaired. In contrast, acrylamide-induced axonopathy begins with degeneration of the distal end of the axon in conjunction with damage to retrograde transport. The primary point of action of some other neurotoxins such as colchicines and paclitaxel (Taxol) is microtubule-based transport along the axons. Taxol binds to the tubuli and colchicine binds to monomeric tubulin. In this way they disturb the dynamic equilibrium of formation and degradation of the microtubules.

Myelinopathies are neurotoxic diseases based on damage to myelin. Myelin, which is formed by the oligodendrocytes in the central nervous system (CNS) and by the Schwann cells in the peripheral nervous system (PNS), is necessary for efficient impulse conduction along the axons. Whereas the Schwann cells of the PNS make it possible for myelin to be regenerated after neurotoxic damage, remyelination is only possible to a limited extent in the CNS. Hexachlorophene, for example, binds firmly to cell membranes and leads to a loss of the ion gradient and finally to edema between the myelin layers. The symptoms of acute hexachlorophene intoxication start with general weakness, irritations and spasms, ultimately leading to coma and death.

In neurotransmission-associated neurotoxicity, primarily the process of neurotransmission is impaired. The neurotoxins of this subclass interrupt impulse conduction, block or intensify trans-synaptic communication, block reuptake of the neurotransmitter or interfere with the “second messenger” system. In most cases these neurotoxins display short-term, reversible interactions, which subside after acute exposure, or can be counteracted with suitable antagonists. In chronic exposure, however, there may also be irreversible long-term consequences. Examples of some neurotoxins of this subgroup are given below.

Nicotine, for example, binds agonistically to certain cholinergic receptors. Small doses of nicotine lead to accelerated heartbeat, raised blood pressure and narrowing of peripheral blood vessels. Acute poisoning with nicotine results in sudden overstimulation of the nicotinergic receptors followed by paralysis of the ganglia, which can lead to respiratory arrest.

The euphoriant and habit-forming property of cocaine can be attributed to changes in catecholaminergic neurotransmission. It is mainly dopaminergic neurotransmission that is intensified by blocking of the “dopamine reuptake transporter”. Cocaine abuse is associated with increased risk of cerebrovascular diseases, cerebral perfusion defects and cerebral atrophy. Chronic cocaine consumption is apparently associated with neurodegenerative changes in the striatum, which is probably the cause of the neurological and psychiatric symptoms.

Like cocaine, the amphetamines also affect catecholaminergic neurotransmission. The neurotoxicity of the amphetamine derivative 3,4-methylenedioxymethylamphetamine has been much debated recently (MDMA, “Ecstasy”) (R. Mathias, NIDA Notes Vol. 14, No. 4 “Ecstasy damages the brain and impairs memory in humans” (1999)). This drug stimulates the release of serotonin and leads to a psychedelic state. In addition, the need for food, drink and sleep is suppressed. The acute toxic effects of MDMA include nausea, chills, hallucinations, raised body temperature, trembling, muscle cramps and blurred vision. An overdose leads to high blood pressure, tiredness and panic attacks, and in more severe cases to unconsciousness, convulsions and dramatically raised body temperature. Cardiac failure and heat stroke may develop as a result of an overdose. As well as this acute neurotoxicity, however, it can be assumed there will also be chronic neurotoxic sequelae. There are indications that regular use of MDMA leads to damage of the serotonin-releasing neurons. This is accompanied by significant impairment of memory. There is probably also disturbance of other serotonin-dependent brain functions, such as mood and the sleep cycle. Animal experiments suggest that the damage to the neurons lasts many years, and may even be permanent.

The aim of the present invention is to provide novel, effective medicinal products for the treatment of coma and/or neurotoxicity.

This aim is achieved through the use of at least one colony stimulating factor for the production of a medicinal product for the treatment or prophylaxis of coma and/or neurotoxicity.

Preferably this CSF is a colony stimulating factor from the group G-CSF, M-CSF and GM-CSF. Especially preferably, G-CSF and/or GM-CSF are used as the therapeutic active substance. In particular, human polypeptides, i.e. human G-CSF and/or GM-CSF in the form of recombinant proteins, are used as the therapeutic active substances.

The term “colony stimulating factor” also includes variants of the aforementioned specific factors. These can be homologous, orthologous or paralogous sequences. Said variants include sequences that have at least one base substitution, a base addition or a base deletion, and the variants should always be a polypeptide with the aforementioned biological activity of the respective starting sequence. Functionally homologous variants and derivatives are also included, e.g. PEGylated polypeptides or polypeptides for which the activity of the colony stimulating factors has been improved or prolonged in some other manner.

Polypeptides that are at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%, or to some other percentage stated herein, identical to an amino acid sequence of one of the aforementioned specific colony stimulating factors and where the polypeptide has the respective biological activity of the starting sequence, are also included.

The percentage of identical amino acids preferably relates to a sequence segment of at least 50% of the sequences to be compared and especially preferably over the entire length of the sequences to be compared. A large number of programs that implement algorithms for said comparisons are described in the prior art and are commercially available. Reference may be made in particular to the algorithms of Needleman and Wunsch or Smith and Waterman, which provide especially reliable results. These algorithms can preferably be implemented by means of the following programs: PileUp (J. Mol. Evolution., 25, 351-360, 1987, Higgins et al., CABIOS, 1989: 151-153), Gap and BestFit (Needleman and Wunsch (J. Mol. Biol. 48; 443-453 (1970)) and Smith and Waterman (Adv. Appl. Math. 2: 482-489 (1981))), as part of the GCG software [Genetics Computer Group, 575 Science Drive, Madison, Wis., USA 53711 (1991)]. Especially preferably, the percentage (%) of sequence identity is determined, within the scope of the present invention, with the GAP program over the complete sequence with the following established values: Gap Weight: 50, Length Weight 3, Average Match: 10.000 and Average Mismatch: 0.000.

A polypeptide that only comprises a fragment of the aforementioned colony stimulating factors is also a polypeptide according to the invention. The fragment should then encode a polypeptide that has the biological activity of the starting polypeptide. Said polypeptides comprise or therefore consist of domains of the aforementioned specific polypeptides (starting polypeptides), which impart the biological activity. A fragment in the sense of the invention preferably comprises at least 20, at least 30, at least 50, at least 80, at least 100 or at least 150 successive amino acids of an amino acid sequence of one of the aforementioned colony stimulating factors.

The variants according to the invention preferably have at least 10%, at least 20%, at least 30%, at least 40%, at least 50%, at least 60%, at least 70%, at least 80% or at least 90% of the respective biological activity of the starting polypeptide. The variants according to the invention can, however, also have improved activity relative to the starting polypeptide.

The invention relates moreover to the preferred use according to the invention of colony stimulating factors for the production of a medicinal product for the treatment of the coma states apallic syndrome, “locked-in” syndrome and akinetic mutism.

A further object of the invention comprises the use of colony stimulating factors for the production of a medicinal product for the treatment of neurotransmission-associated neurotoxicity. In particular, said neurotoxicity can have been induced by amphetamine or its derivatives (in particular 3,4-methylenedioxymethylamphetamine).

The CSFs can be administered in a variety of forms, including, among others: solutions for infusion or injection, suspensions, tablets, pills, powders, sprays or suppositories. The preferred form depends on the method of administration and the therapeutic application. The method of administration can be, among others: oral, subcutaneous, pulmonary, intranasal, intramuscular, rectal, intracerebral or intravenous administration. The preferred method of administration is intravenous injection or infusion.

The therapeutically effective dose of the CSFs, which can be administered either alone or as a combination of various CSFs, should be chosen so that a neuroprotective effect is achieved. Therefore the dose can in particular be in a range between 100 ng and 10 mg/kg body weight. By taking into account factors such as the patient's age, sex and severity of the neurological disturbance and selection of the CSF or CSFs used can lead to individually tailored doses. A further modification of the dose can follow from the method of administration and the associated pharmacokinetics and local availability. For example, the dose would be lower in the case of direct intracerebral injection. In certain cases of treatment of the neurological disorders described here, the use of high doses of CSF (e.g. more than 1 mg/kg body weight) can be especially useful.

The treatment is preferably started within the first week after onset of the neurological disturbance, but later commencement of treatment is also possible with these often chronic neurological disorders. For treating said chronic forms of neurological disorders, a regular, preferably daily, dose of CSF can be administered. It can then preferably be administered in a formulation that permits slow, continuous release of the active substance (“slow-release formulation”). This slow, continuous administration of the active substance can also be achieved e.g. by infusion or using micrometering pumps.

The pharmacological preparations with one or more CSFs as active substance can be prepared by the standard methods from the prior art that are known by a person skilled in the art. Pharmaceutically acceptable excipients can be added to the preparation. The appropriate form of the pharmacological preparation and the method of administration can be selected in relation to the neurological disturbance to be treated, its severity and other relevant circumstances. The pharmacological preparation can be adapted for oral, parenteral or topical administration. The CSFs used as the active substance can also be used in the form of a pharmacologically acceptable salt, e.g. for reasons of stability, solubility or better crystallizability.

1.-8. (canceled)

9. A method for the treatment or prophylaxis of neurotoxicity, wherein a therapeutically effective dose of G-CSF, GM-CSF or of a polypeptide with an identity of at least 90% to either G-CSF or GM-CSF is administered to a patient in need thereof.

10. The method as claimed in claim 9, wherein the G-CSF is human G-CSF.

11. The method as claimed in claim 9, wherein the GM-CSF is human GM-CSF.

12. The method as claimed in claim 9, wherein the neurotoxicity is a neurotransmission-associated neurotoxicity.

13. The method as claimed in claim 9, wherein the neurotoxicity is a neurotoxicity induced by amphetamine or derivatives thereof.

14. The method as claimed in claim 13, wherein the amphetamine derivative is a 3,4-methylenedioxymethylamphetamine.
